(19) United States
(12) Patent Application Publication
(10) Pub. No.: US 2024/0115289 A1
(43) Pub. Date: Apr. 11, 2024

(54) ROTATIONAL DRIVE SHAFTS AND INTRAVASCULAR MEDICAL DEVICES THEREOF

(71) Applicant: Cardiovascular Systems, Inc.

(72) Inventors: Jacob P. Draxler, St. Paul, MN (US); Nicholas Ellering, Crystal, MN (US); Huisun Wang, Maple Grove, MN (US)

(21) Appl. No.: 17/938,490

(22) Filed: Oct. 6, 2022

(51) Int. Cl. A61B 17/3207

(52) U.S. Cl. CPC A61B 17/320758; A61B 2017/00853

(57) ABSTRACT

Devices and systems comprising a rotational drive shaft formed of wire filars or one or more coils for use in high-speed rotational medical procedures, e.g., atherectomy, are disclosed. Generally, a preferred embodiment of the drive shaft for transferring torque and activating rotation of a tool such as an abrasive element that is attached near a distal end of the drive shaft may be constructed with a heat shrinkable polymer layer covering at least a middle portion of the drive shaft, wherein a proximal-most portion of the drive shaft is not covered by the heat shrinkable polymer layer. In certain embodiments, the drive shaft may also comprise a distal portion that is not covered by the heat shrinkable layer, most preferably the distal end of the heat shrinkable layer in this embodiment is configured to remain within a delivery catheter or sheath during a medical procedure.

### CROSS-REFERENCE TO RELATED APPLICATIONS

None

### STATEMENT REGARDING FEDERALLY SPONSORED RESEARCH OR DEVELOPMENT

Not Applicable

### BACKGROUND OF THE INVENTION

### Field of the Invention

The disclosure relates to devices and methods using guidewires, for example intravascular procedures, e.g., removing tissue from body passageways, such as removal of atherosclerotic plaque from arteries with, e.g., a rotational atherectomy device. More specifically, the disclosure provides a guidewire tip that is reformable and, therefore, resistant to damaging deformation.

### Description of the Related Art

A variety of techniques and instruments have been developed for use in the removal or repair of tissue in arteries and similar body passageways. A frequent objective of such techniques and instruments is the removal of atherosclerotic plaques in a patient's arteries. Atherosclerosis is characterized by the buildup of fatty deposits (atheromas) in the intimal layer (under the endothelium) of a patient's blood vessels. Very often over time, what initially is deposited as relatively soft, cholesterol-rich atheromatous material hardens into a calcified atherosclerotic plaque. Such atheromas restrict the flow of blood, and therefore often are referred to as stenotic lesions or stenoses, the blocking material being referred to as stenotic material. If left untreated, such stenoses can cause angina, hypertension, myocardial infarction, strokes and the like.

Rotational atherectomy procedures have become a common technique for removing such stenotic material. Such procedures are used most frequently to initiate the opening of calcified lesions in coronary arteries. Most often the rotational atherectomy procedure is not used alone, but is followed by a balloon angioplasty procedure, which, in turn, is very frequently followed by placement of a stent to assist in maintaining patency of the opened artery. For non-calcified lesions, balloon angioplasty most often is used alone to open the artery, and stents often are placed to maintain patency of the opened artery. Studies have shown, however, that a significant percentage of patients who have undergone balloon angioplasty and had a stent placed in an artery experience stent restenosis, which is blockage of the stent that most frequently develops over a period of time as a result of excessive growth of scar tissue within the stent. In such situations an atherectomy procedure is the preferred procedure to remove the excessive scar tissue from the stent (balloon angioplasty being not very effective within the stent), thereby restoring the patency of the artery.

In one type of rotational atherectomy device, such as that shown in U.S. Pat. No. 4,990,134 (Auth), a burr covered with an abrasive abrading material such as diamond particles is carried at the distal end of a flexible drive shaft. The burr is rotated at high speeds (typically, e.g., in the range of about 150,000-190,000 rpm) while it is advanced across the stenosis. As the burr is removing stenotic tissue, however, it blocks blood flow. Once the burr has been advanced across the stenosis, the artery will have been opened to a diameter equal to or only slightly larger than the maximum outer diameter of the burr. Frequently more than one size burr must be utilized to open an artery to the desired diameter.

U.S. Pat. No. 5,314,438 (Shturman) discloses another atherectomy device having a drive shaft with a section of the drive shaft having an enlarged diameter, at least a segment of this enlarged surface being covered with an abrasive material to define an abrasive segment of the drive shaft. This system may be referred to as an orbital atherectomy device or system (“OAD”). When rotated at high speeds, the abrasive segment is capable of removing stenotic tissue from an artery. Though this atherectomy device possesses certain advantages over the Auth device due to its flexibility, it also is capable only of opening an artery to a diameter about equal to the diameter of the enlarged abrading surface of the drive shaft since the device is not eccentric in nature.

U.S. Pat. No. 6,494,890 (Shturman) discloses a known OAD having a drive shaft with an enlarged eccentric section, wherein at least a segment of this enlarged section is covered with an abrasive material. When rotated at high speeds, the abrasive segment is capable of removing stenotic tissue from an artery. The device is capable of opening an artery to a diameter that is larger than the resting diameter of the enlarged eccentric section due, in part, to the orbital rotational motion during high speed operation. Since the enlarged eccentric section comprises drive shaft wires that are not bound together, the enlarged eccentric section of the drive shaft may flex during placement within the stenosis or during high speed operation. This flexion allows for a larger diameter opening during high speed operation, but may also provide less control than desired over the diameter of the artery actually abraded. In addition, some stenotic tissue may block the passageway so completely that the Shturman device cannot be placed therethrough. Since Shturman requires that the enlarged eccentric section of the drive shaft be placed within the stenotic tissue to achieve abrasion, it will be less effective in cases where the enlarged eccentric section is prevented from moving into the stenosis.

Shturman further teaches that a portion of the drive shaft that is proximal to the eccentric enlarged diameter section may be encased in a thin, flexible, low friction sheath or coating. In a preferred embodiment, Shturman teaches that the sheath or coating is sufficiently long so that its proximal end remains disposed inside the catheter even when the drive shaft, with its enlarged diameter section, is fully advanced distally with respect to the catheter. Shturman also teaches that, as evidenced by FIGS. 23A-23C, illustrated herein as prior art FIG. 1, that the sheath or coating effectively creates a smoothed, non-undulating covering around the wire filars of the drive shaft. Even though the sheath or coating may comprise a heat shrinkable material, it is clear from FIG. 1 that the coating or sheath is not sufficiently tightened around the wire filars of the drive shaft to create undulations along the drive shaft length, but instead creates a smoothed outer profile, that is, without undulations.

The disclosure of U.S. Pat. No. 6,494,890 is hereby incorporated by reference in its entirety.

Moreover, we provide disclosure of the following patents and applications, each of which are assigned to Cardiovascular Systems, Inc., and incorporated herein in their entirety, each of which may comprise systems, methods and/or devices that may be used with various embodiments of the presently disclosed subject matter:
U.S. Pat. No. 6,295,712, “ROTATIONAL ATHERECTOMY DEVICE”;
U.S. Pat. No. 6,494,890, “ECCENTRIC ROTATIONAL ATHERECTOMY DEVICE”;
U.S. Pat. No. 6,132,444, “ECCENTRIC DRIVE SHAFT FOR ATHERECTOMY DEVICE AND METHOD FOR MANUFACTURE”;
U.S. Pat. No. 6,638,288, “ECCENTRIC DRIVE SHAFT FOR ATHERECTOMY DEVICE AND METHOD FOR MANUFACTURE”;
U.S. Pat. No. 5,314,438, “ABRASIVE DRIVE SHAFT DEVICE FOR ROTATIONAL ATHERECTOMY”;
U.S. Pat. No. 6,217,595, “ROTATIONAL ATHERECTOMY DEVICE”;
U.S. Pat. No. 5,554,163, “ATHERECTOMY DEVICE”;
U.S. Pat. No. 7,507,245, “ROTATIONAL ANGIOPLASTY DEVICE WITH ABRASIVE CROWN”;
U.S. Pat. No. 6,129,734, “ROTATIONAL ATHERECTOMY DEVICE WITH RADIALLY EXPANDABLE PRIME MOVER COUPLING”;
U.S. patent application Ser. No. 11/761,128, “ECCENTRIC ABRADING HEAD FOR HIGH-SPEED ROTATIONAL ATHERECTOMY DEVICES”;
U.S. patent application Ser. No. 11/767,725, “SYSTEM, APPARATUS AND METHOD FOR OPENING AN OCCLUDED LESION”;
U.S. patent application Ser. No. 12/130,083, “ECCENTRIC ABRADING ELEMENT FOR HIGH-SPEED ROTATIONAL ATHERECTOMY DEVICES”;
U.S. patent application Ser. No. 12/363,914, “MULTI-MATERIAL ABRADING HEAD FOR ATHERECTOMY DEVICES HAVING LATERALLY DISPLACED CENTER OF MASS”;
U.S. patent application Ser. No. 12/578,222, “ROTATIONAL ATHERECTOMY DEVICE WITH PRE-CURVED DRIVE SHAFT”;
U.S. patent application Ser. No. 12/130,024, “ECCENTRIC ABRADING AND CUTTING HEAD FOR HIGH-SPEED ROTATIONAL ATHERECTOMY DEVICES”;
U.S. patent application Ser. No. 12/580,590, “ECCENTRIC ABRADING AND CUTTING HEAD FOR HIGH-SPEED ROTATIONAL ATHERECTOMY DEVICES”;
U.S. patent application Ser. No. 29/298,320, “ROTATIONAL ATHERECTOMY ABRASIVE CROWN”;
U.S. patent application Ser. No. 29/297,122, “ROTATIONAL ATHERECTOMY ABRASIVE CROWN”;
U.S. patent application Ser. No. 12/466,130, “BIDIRECTIONAL EXPANDABLE HEAD FOR ROTATIONAL ATHERECTOMY DEVICE”;
U.S. patent application Ser. No. 12/388,703, “ROTATIONAL ATHERECTOMY SEGMENTED ABRADING HEAD AND METHOD TO IMPROVE ABRADING EFFICIENCY”; and
U.S. patent application Ser. No. 13/624,313, “ROTATIONAL ATHERECTOMY DEVICE WITH ELECTRIC MOTOR”.

The prior art is vulnerable to the sudden release of, e.g., an atherectomy tool that is at least partially blocked or stuck within an occlusion. Release of the tool allows the drive shaft to return to an undeformed, or unwound, position which may result in a sudden change in axial position within the subject vessel.

It is, therefore, desirable to provide a rotational drive shaft for intravascular medal devices that, unlike the above-referenced art, comprises a middle portion that comprises greater rotational stiffness than a proximal and/or distal portion of the drive shaft. This structure is desirable as it provides superior torque strain relief function and aid in preventing drive shaft filar or coil fracture during blockage episodes.

The various embodiments described herein address these, inter alia, issues.

### BRIEF SUMMARY OF THE INVENTION

Various embodiments of devices and systems comprising a rotational drive shaft formed of wire filars or one or more coils for use in high-speed rotational medical procedures, e.g., atherectomy, are disclosed. Generally, a preferred embodiment of the drive shaft for transferring torque and activating rotation of a tool attached thereto, e.g., an abrasive element, may be constructed with a heat shrinkable polymer layer covering at least a middle portion of the drive shaft, wherein a proximal-most portion of the drive shaft is not covered by the heat shrinkable polymer layer. In certain embodiments, the drive shaft may also comprise a distal portion that is not covered by the heat shrinkable layer, most preferably the distal end of the heat shrinkable layer in this embodiment is configured to remain within a delivery catheter or sheath during a medical procedure.

In certain embodiments, the heat shrinkable layer over at least the middle portion of the drive shaft's length is tightly formed against the wire filars and/or coil(s) of the drive shaft such that the heat shrinkable layer comprises or forms or defines undulations along the length of the drive shaft that is covered by the heat shrinkable layer. These embodiments work to tightly hold the wire filars and/or coil(s) of the drive shaft with an inwardly directed radial compression force that restricts longitudinal elongation of the covered portion of the wire filars and/or coil(s) of the drive shaft during high-speed rotation and/or instances where the tool becomes blocked within an occlusion. The proximal uncovered portion of the wire filars and/or coil(s) of the drive shaft provide essential strain relief to aid in reducing excess torque. The length of the proximal uncovered portion is critical in maintaining 1:1 motion control (longitudinal jump reduction control) vs. provision of the strain relief function.

The figures and the detailed description which follow more particularly exemplify these and other embodiments of the invention.

### BRIEF DESCRIPTION OF THE SEVERAL VIEWS OF THE DRAWINGS

FIG. 1 is a series of three cutaway longitudinal views of one embodiment of a known rotational atherectomy device and system;

FIG. 2 is a perspective view of one embodiment of a known rotational atherectomy device and system;

FIG. 3 is a side and cutaway view of one embodiment of the present invention; and

FIG. 4 is a side and partial cutaway view of one embodiment of the present invention.

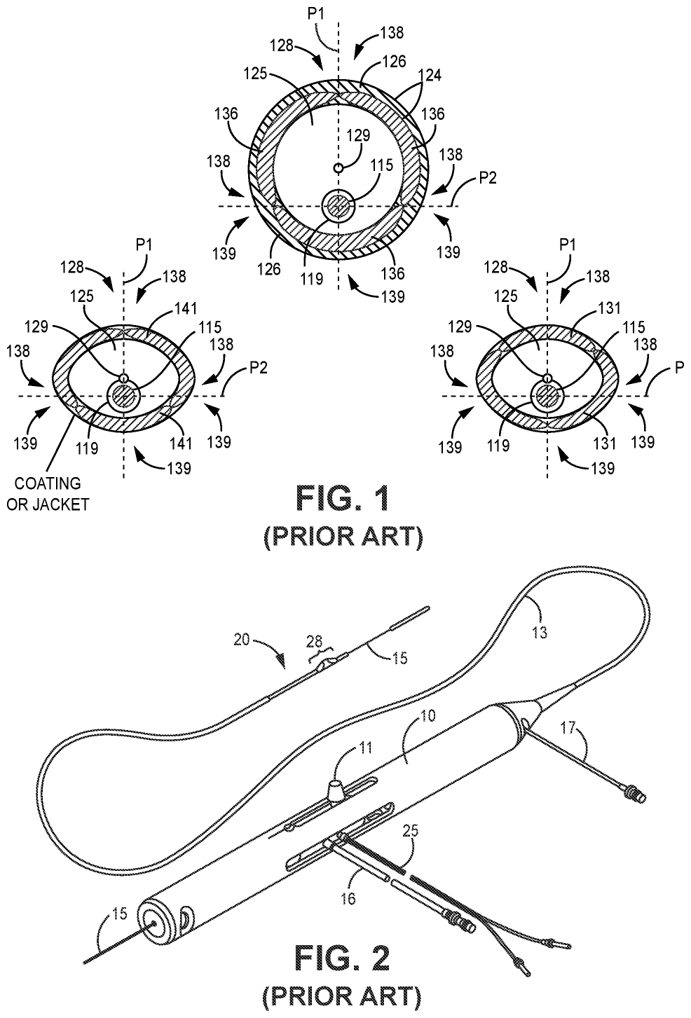

### DETAILED DESCRIPTION OF THE INVENTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

FIG. 2 illustrates one known embodiment of an exemplary rotational atherectomy device or OAD. The device includes a handle portion 10, an elongated, flexible drive shaft 20 having an eccentric enlarged abrading head 28, and an elongated catheter 13 extending distally from the handle portion 10. The drive shaft 20 is constructed from helically coiled wire as is known in the art and an abrasive element 28 is fixedly attached thereto. Known drive shafts are manufactured from a multi-filar wound coil. The inherent construction of this known coil allows for spaces between the filars. These spaces allow fluid, e.g., saline and/or water or other fluid, to pass through to the inner diameter of the drive shaft coil in order to provide cooling and/or lubrication effects to the interface of the drive shaft coil and the guide wire 15.

Generally, in this known construction, the proximal end of the coiled drive shaft is rotationally coupled with a prime mover, thereby providing a rotational connection between the prime mover, e.g., an electric motor or turbine, and the drive shaft 20. In the known systems, the prime mover is disposed within handle 10.

Continuing with reference to FIG. 2, the known catheter 13 has a lumen in which most of the length of the drive shaft 20 is disposed, except for the enlarged abrading head 28 and a short section distal to the abrasive element 28. The drive shaft 20 also contains an inner lumen, permitting the drive shaft 20 to be advanced and rotated over a guide wire 15. A fluid supply line 17 may be provided for introducing a cooling and lubricating solution (typically saline or another biocompatible fluid) into the catheter 13.

The handle 10 desirably contains a rotational drive mechanism or prime mover for rotating the drive shaft 20 at high speeds. The handle 10 typically may be connected to a power source to power the prime mover, such as an electrical power source, or compressed air delivered through a tube 16. A pair of fiber optic cables 25, alternatively a single fiber optic cable may be used, may also be provided for monitoring the speed of rotation of the turbine and drive shaft 20. The handle 10 also desirably includes a control knob 11 for advancing and retracting the prime mover and/or drive shaft 20 with respect to the catheter 13 and the body of the handle.

Embodiments of the drive shaft disclosed herein may comprise at least one tool, typically near the distal end of the drive shaft such as an abrasive element 28 as shown in FIG. 2. An exemplary tool comprises an atherectomy tool such as one or more burrs or abrasive crowns or abrasive elements, and which may be concentric with center of mass located on the longitudinal axis of the drive shaft, and/or eccentric with center of mass located radially off of the longitudinal axis of the drive shaft. Other tools may comprise sensors, transceivers, receivers and the like for, inter alia, obtaining, sending and/or receiving data regarding the biological lumen such as position/distance, elastance, compliance, composition, etc., that the drive shaft is received within and/or the region of interest, e.g., a lesion, within the lumen.

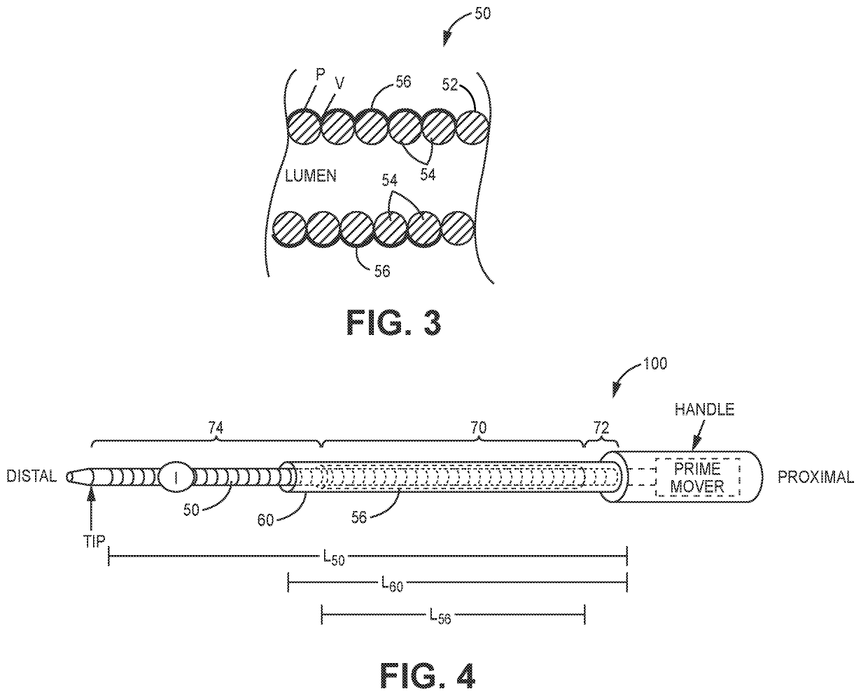

Turning now to FIG. 3, a cross-section of a drive shaft 50 is illustrated as comprising a coil formed of wire filars 54 in a substantially circular coil 52 defining a lumen therethrough. The polymer coating or jacket 56 is provided on an outer surface of the wire filars 54 and tightly formed against the wire filars 54 such that a recurring series of alternating peaks P and valleys V are defined and which collectively form undulations along the length of the coated and restrained portion of the drive shaft 50. The polymer or coating 56 is tightly formed around the wire filars 54 as shown to restrain individual wire filars 54 from moving in the longitudinal or axial direction relative to each other as well as moving or expanding radially. The polymer jacket or coating 56 generates an inwardly directed or radial compression force against the wire filars 54 of the drive shaft 50 which is sufficient to prevent longitudinal lengthening or shortening of the portion of the drive shaft 50 covered by the polymer coating or jacket 56.

The polymer coating or jacket 56 is applied, in certain embodiments, as a heat shrinkable material such as heat-shrink tube materials, such as FEP (fluorinated ethylene propylene), PTFE (Polytetrafluoroethylene), polyolefin, polyvinyl chloride, elastomeric heat shrink tubing, polyvinylidene fluoride (PVDF), silicone, synthetic rubber such as fluroelastomer, and other thermally deformable or shrinkable materials and may not comprise a smooth or linear profile in the longitudinal direction. As seen in FIG. 3, undulations may be formed or defined by the polymer coating or jacket 56 with peaks P and valleys V that form around the individual filars 54. This configuration provides additional surface area of the wire filars 54 to be adhered and/or connected with the conforming polymer coating or jacket 56 which further aids in increased radial compression force to be applied to the wire filars 56 and/or coil 52 and concomitant reduced or restricted longitudinal expansion or lengthening, or longitudinal compression or shortening, of the covered wire filars 54 during high speed rotation. As can be seen in FIG. 3, the polymer coating or jacket 56 covers an increased radius of each covered filar 54, including the regions defined by the valleys V which, in turn, results in increased surface area connection between the polymer coating or jacket 56 and the covered section of the drive shaft filars 54. The increased surface area of the filars 54 that is covered by the polymer coating or jacket 56 allows for additional regions of connection therebetween and additional regions to apply inwardly radially compressing force as well as axially or longitudinally restricting force to the covered filars 54. In other embodiments, the outer profile of the polymer coating or jacket 56 is substantially free of undulations while still providing the necessary restriction of longitudinal lengthening and/or shortening of the covered portion of the drive shaft. Stated differently, the tightly conforming polymer coating or jacket 56 constrains the wire filars 54 of the drive shaft 50 in the longitudinal or axial direction or dimension as well as in the radial direction or dimension by restricting or preventing radial and/or axial movement of the covered filars 56. Accordingly, at least the section of the drive shaft 50 that is covered by the conforming and, in certain embodiments undulating, polymer coating or jacket 56 is constrained from lengthening, or shortening, as well as expanding to achieve a larger diameter than a nominal diameter or compressing to achieve a smaller diameter than the nominal diameter.

Manufacturing the conforming and undulating polymer coating or jacket 56 may be achieved as follows:
1. Securing the coil(s) 52 comprising wire filars 54 over a mandrel.
2. Securing the coil(s) 52 in a straightened and fully compressed configuration.
3. Placing a polymer heat shrinkable tube over the straightened and fully compressed coil(s) 52, wherein the polymer tube has a length that is less than the length of the coil(s) and wherein a proximal and distal sections of the coil are not covered by the polymer heat shrinkable tube.
4. Applying heat to shrink both ends of the polymer heat shrinkable tube around the straightened and fully compressed coil(s) 52.
5. Placing the coil(s) 52 with the polymer heat shrinkable tube into an oven set between 120 degrees C. to 160 degrees C. for a period of 5 to 10 minutes.
6. Removing the coil(s) 52 with fully constraining polymer coating and or jacket 56.
7. Cooling the resulting drive shaft 50 with constraining and undulating polymer coating and/or jacket 56.
8. Releasing the coil(s) 52 from the fully compressed configuration.

In certain embodiments, the fully compressed coil(s) 52 that are now coated and restrained by the undulating polymer coating and/or jacket 56 by the above process will remain in a fully compressed configuration after application and coating of the polymer thereto and the coil(s) 56 are released from the full compression of step 2. In other embodiments, the coils(s) 52 coated and restrained with the undulating polymer coating may remain in an at least partially compressed configuration after application and coating of the polymer thereto and the coil(s) 56 are released from the full compression of step 2.

FIG. 4 illustrates an exemplary rotational atherectomy device 100 with an embodiment of the drive shaft 50 with a constrained portion 70. A proximally located handle with prime mover incorporated therein is rotationally connected with a proximal end of the drive shaft 50. An outer sheath 60, generally for providing space around the drive shaft 50 to deliver saline, extends distally away from the handle and comprises a lumen therethrough within which the drive shaft 60 extends. The drive shaft 50 is configured to rotate and translate within the lumen of the outer sheath 60.

The outer sheath 60 comprises a length L60 and a distal end, wherein the drive shaft 50 extends distally beyond the distal end of the outer sheath 60 such that the length L50 of the drive shaft 50 is greater than the length L60 of the outer sheath 60.

The conforming and undulating polymer coating or jacket 56 comprises a length L56 that is less than the length L 60 of the outer sheath 60 and less than the length L50 drive shaft 50, and therefore comprises a distal end that is configured to be located proximal to the distal end of the outer sheath 60. The conforming and undulating polymer coating or jacket 56 is in preferred embodiments disposed entirely within the lumen formed by outer sheath 60 and defines a constrained portion of the drive shaft 70. However, in other embodiments, the polymer coating or jacket 56 may extend distally beyond the distal end of the outer sheath 60.

In addition, an unconstrained proximal-most portion of the drive shaft 72 is provided adjacent to the connection of the handle and the drive shaft and extends distally away from the handle. Accordingly, the proximal end of the polymer coating or jacket is spaced longitudinally away from the handle in the distal direction and the entire portion of the drive shaft that is covered by the polymer coating or jacket is disposed within the lumen of the outer sheath.

In addition, in preferred embodiments, an unconstrained distal portion of the drive shaft 74, including, but in some embodiments not limited to, the portion extending distally away from the distal end of the outer sheath 60, is provided and is not constrained by the polymer coating or jacket 56.

The exemplary tool T disposed near the distal end of the drive shaft is shown as an eccentric abrasive element with a center of mass radially offset from the longitudinal axis of the drive shaft 50. An atraumatic tip is provided at the distal end of the drive shaft 50

The unconstrained proximal-most section 72 of the drive shaft 50 provides a strain relief function and aids in avoiding non-elastic deformation of the drive shaft wire filars 54 and/or coil(s) 52 and, in extreme cases, avoids shaft fracture when the drive shaft 50 become blocked during high-speed rotation. At the same time, the constrained portion 70 of the drive shaft 50 in combination with the unconstrained proximal-most section 72 of the drive shaft 50 provides 1:1 motion control, or “jump” mitigation. Jump as used herein is a term used to describe the resulting change of the drive shaft's length and/or radial diameter following a sudden release of a blocked drive shaft 50 under torqueing force, and/or that is at least partially wound or elastically deformed with related potential energy storage, that has been at least partially blocked within an occlusion. The wire filars 54 and/or coil(s) 52 will, upon release from the at least partial blocking condition, seek to return to a non-deformed state. The resultant “jump” of the unblocked drive shaft 50 will cause the drive shaft 50 and tool T disposed thereon to quickly move axially. This phenomenon occurs very rapidly and can be a source of trauma within the vasculature.

The length of the unconstrained proximal-most section 72 of the drive shaft 50 is a critical feature that balances the above-referenced functions and may be within the range of 0.5-3.0 inches, with 1-2 inches being preferred. If the length of the unconstrained proximal-most uncovered section 72 is longer than the stated ranges, the 1:1 motion control or “jump” reduction of the drive shaft 50 may be reduced to an unsafe level. If the unconstrained proximal-most uncovered section 72 is too short, i.e., less than the stated ranges, then the necessary strain relief function is not provided and can result in non-elastic deformation and/or fracture of the wire filars 54 and/or coil(s) 52. Thus, the stated ranges strike the necessary balance for maintaining 1:1 motion control over the rotational position of the drive shaft 50 and related tool T while providing the requisite strain relief.

Further, an exemplary drive shaft length L50 may be approximately 57 inches. In certain embodiments, the length of the unconstrained proximal-most section 72 may be approximately 1.2 inches which is approximately 2.1% of the overall drive shaft length L50. Further, in certain embodiments, the length of the unconstrained distal section 74 may be approximately 4.8 inches in length which is approximately 8.38% of the total drive shaft length L50.

Thus, the following percentage of the unconstrained proximal-most section 72 relative to the total drive shaft length L50 may be applied to any length of drive shaft as follows:

Percentage of the unconstrained proximal-most section 72 is within 0.8%-5.2% of the total drive shaft length L50.

A preferred percentage of the unconstrained proximal-most section 72 is within 1.8%-3.5% of the total drive shaft length L50.

The descriptions of the embodiments and their applications as set forth herein should be construed as illustrative, and are not intended to limit the scope of the disclosure. Features of various embodiments may be combined with other embodiments and/or features thereof within the metes and bounds of the disclosure. Upon study of this disclosure, variations and modifications of the embodiments disclosed herein are possible, and practical alternatives to and equivalents of the various elements of the embodiments will be understood by and become apparent to those of ordinary skill in the art. Such variations and modifications of the embodiments disclosed herein may be made without departing from the scope and spirit of the invention. Therefore, all alternatives, variations, modifications, etc., as may become to one of ordinary skill in the art are considered as being within the metes and bounds of the instant disclosure.

What is claimed is:

1. A rotational atherectomy device comprising:
a handle comprising a prime mover;
an elongate drive shaft comprising wire filars and rotationally connected with the prime mover comprising:
a constrained drive shaft section wherein the wire filars of the constrained drive shaft section are constrained in an at least partially longitudinally compressed configuration by a heat shrinkable polymer coating,
an unconstrained proximal-most drive shaft section disposed proximal to the constrained drive shaft section, wherein the proximal-most drive shaft section is not covered or constrained by the heat shrinkable polymer coating, and
an unconstrained distal-most drive shaft section disposed distal to the constrained drive shaft section, wherein the distal-most drive shaft section is not covered or constrained by the heat shrinkable polymer coating.

2. The rotational atherectomy device of claim 1, wherein a total length of the drive shaft as measured from the handle is approximately 57 inches, and a length of the unconstrained proximal-most drive shaft section is within the range of 0.5 to 3 inches.

3. The rotational atherectomy device of claim 1, wherein a total length of the drive shaft as measured from the handle is approximately 57 inches, and a length of the unconstrained proximal-most drive shaft section is within the range of 1 to 2 inches.

4. The rotational atherectomy device of claim 1, wherein the drive shaft comprises a length measured from the handle, and a length of the unconstrained proximal-most drive shaft section as a percentage of the drive shaft length is within the range of 0.8% to 5.2%.

5. The rotational atherectomy device of claim 1, wherein the drive shaft comprises a length measured from the handle, and a length of the unconstrained proximal-most drive shaft section as a percentage of the drive shaft length is within the range of 1.8% to 3.5%.

6. The rotational atherectomy device of claim 1, further comprising an outer sheath extending distally away from the handle and comprising a lumen defined therethrough and a length,
wherein the drive shaft extends through the lumen of the outer sheath, and
wherein the length of the outer sheath is less than a length of the constrained drive shaft section.

7. The rotational atherectomy device of claim 1, further comprising an outer sheath extending distally away from the handle and comprising a lumen defined therethrough and a distal end,
wherein the drive shaft extends through the lumen of the outer sheath, and
wherein the distal end of the outer sheath is located distal to a distal end of the constrained drive shaft section.

8. The rotational atherectomy device of claim 1, further comprising an outer sheath extending distally away from the handle and comprising a lumen defined therethrough and a distal end,
wherein the drive shaft extends through the lumen of the outer sheath, and
wherein the distal end of the outer sheath is spaced distally away from a distal end of the constrained drive shaft section.

9. The rotational atherectomy device of claim 8, wherein the unconstrained proximal-most drive shaft section is located within the lumen of the outer sheath.

10. The rotational atherectomy device of claim 1, wherein the heat shrinkable polymer coating partially conforms with the wire filars of the drive shaft and defines an undulating longitudinal profile consisting of alternating peaks and valleys.

11. The rotational atherectomy device of claim 1, wherein the wire filars of the constrained drive shaft section are constrained in a fully longitudinally compressed configuration by the heat shrinkable polymer coating,

12. The rotational atherectomy device of claim 1, further comprising at least one tool disposed at or near a distal end of the drive shaft within the distal-most drive shaft section that is not constrained by the heat shrinkable polymer coating.

13. An elongate drive shaft comprising wire filars comprising wire filars and rotationally connected with a prime mover that is disposed within a handle, the elongate drive shaft comprising:
a constrained drive shaft section wherein the wire filars of the constrained drive shaft section are constrained in an at least partially longitudinally compressed configuration by a heat shrinkable polymer coating,
an unconstrained proximal-most drive shaft section disposed proximal to the constrained drive shaft section, wherein the proximal-most drive shaft section is not covered or constrained by the heat shrinkable polymer coating, and
an unconstrained distal-most drive shaft section disposed distal to the constrained drive shaft section, wherein the distal-most drive shaft section is not covered or constrained by the heat shrinkable polymer coating.

14. The elongate drive shaft of claim 13, wherein a total length of the drive shaft as measured from the handle is approximately 57 inches, and a length of the unconstrained proximal-most drive shaft section is within the range of 0.5 to 3 inches.

15. The elongate drive shaft of claim 13, wherein a total length of the drive shaft as measured from the handle is approximately 57 inches, and a length of the unconstrained proximal-most drive shaft section is within the range of 1 to 2 inches.

16. The elongate drive shaft of claim 13, wherein the drive shaft comprises a length measured from the handle, and a length of the unconstrained proximal-most drive shaft section as a percentage of the drive shaft length is within the range of 0.8% to 5.2%.

17. The elongate drive shaft of claim 13, wherein the drive shaft comprises a length measured from the handle, and a length of the unconstrained proximal-most drive shaft section as a percentage of the drive shaft length is within the range of 1.8% to 3.5%.

18. The elongate drive shaft of claim 13, wherein the heat shrinkable polymer coating partially conforms with the wire filars of the drive shaft and defines an undulating longitudinal profile consisting of alternating peaks and valleys.

19. The elongate drive shaft of claim 13, wherein the wire filars of the constrained drive shaft section are constrained in a fully longitudinally compressed configuration by the heat shrinkable polymer coating,

20. The elongate drive shaft of claim 13, further comprising at least one tool disposed at or near a distal end of the drive shaft within the distal-most drive shaft section that is not constrained by the heat shrinkable polymer coating.

21. A method for providing strain relief for a rotational drive shaft, comprising:
providing a high-speed rotational atherectomy system comprising a prime mover in rotational connection with a proximal end of the rotational drive shaft, the prime mover disposed within a handle;
providing the rotational drive shaft with:
a constrained drive shaft section wherein the wire filars of the constrained drive shaft section are constrained in an at least partially longitudinally compressed configuration by a heat shrinkable polymer coating,
a proximal-most drive shaft section disposed proximal to the constrained drive shaft section, wherein the proximal-most drive shaft section is not covered or constrained by the heat shrinkable polymer coating, and
a distal-most drive shaft section disposed distal to the constrained drive shaft section, wherein the distal-most drive shaft section is not covered or constrained by the heat shrinkable polymer coating.

22. A method of making a rotational drive shaft formed from at least one coil comprising wire filars, comprising:
securing the at least one coil comprising wire filars in a straightened and fully longitudinally compressed configuration;
placing a polymer heat shrinkable tube having two ends and a length that is less than the at least one coil over the straightened and fully compressed at least one coil, wherein the polymer heat shrinkable tube is not disposed over a proximal section and a distal section of the at least one coil;
applying heat to shrink both ends of the polymer heat shrinkable tube around the straightened and fully compressed at least one coil;
placing the at least one coil with the polymer heat shrinkable tube into an oven set between 120 degrees C. to 160 degrees C. for a period of 5 to 10 minutes;
removing the at least one coil with fully constraining polymer coating and or jacket disposed therearound;
cooling the resulting drive shaft with constraining and undulating polymer coating and/or jacket; and
allowing the at least one coil to decompress, wherein the section of the at least one coil that is covered by the constraining polymer coating and/or jacket remains at least partially compressed.
